(19) United States
(12) Patent Application Publication
(10) Pub. No.: US 2013/0059929 A1
(43) Pub. Date: Mar. 7, 2013

(54) MICROBIOLOGICALLY STABLE, EASILY APPLICABLE PREPARATIONS

(71) Applicants: Manuela Koehler, Hamburg (DE); Jens Schulz, Rellingen (DE); Alexander Filbry, Hamburg (DE); Rainer Kroepke, Schenefeld (DE); Lara Terstegen, Hamburg (DE); Anke Pilzner, Hamburg (DE); Horst Argembeaux, Wentorf (DE)

(72) Inventors: Manuela Koehler, Hamburg (DE); Jens Schulz, Rellingen (DE); Alexander Filbry, Hamburg (DE); Rainer Kroepke, Schenefeld (DE); Lara Terstegen, Hamburg (DE); Anke Pilzner, Hamburg (DE); Horst Argembeaux, Wentorf (DE)

(73) Assignee: Beiersdorf AG, Hamburg (DE)

(21) Appl. No.: 13/583,174

(22) PCT Filed: Sep. 2, 2010
(86) PCT No.: PCT/EP2010/005375

§ 371 Date: Nov. 12, 2012

(30) Foreign Application Priority Data
Mar. 29, 2010 (DE) 10 2010 013 274.8

(51) Int. Cl. A61K 8/49; A61K 8/43; C07C 217/08; A61Q 19/00; C07D 213/89; C07C 279/14; A61Q 5/02; A61K 8/41; C07D 275/03

(52) U.S. Cl. 514/784; 514/788; 564/287; 548/213; 546/290; 562/560

(57) ABSTRACT

Cosmetic or dermatological preparations show improved sensory properties and sufficient microbiological stability due to the addition of benzethonium chloride, methylisothiazolinone, piroctone olamine and/or lauroyl ethyl arginate, the cosmetic or dermatological preparations not comprising any other preservatives, and are especially free of parabens and/or phenoxyethanol.

The invention comprises benzethonium chloride, methylisothiazolinone, piroctone olamine and/or lauroyl ethyl arginate in cosmetic or dermatological preparations which comprise no further preservatives with phenol groups, in particular are free from parabens and/or phenoxyethanol.

In order that cosmetic or dermatological creams and lotions can be kept for some months after opening, they have to be preserved. The choice of agents is decisive—since bacteria, fungi and yeasts do not differentiate whether a cream or lotion contains natural or synthetic substances. To open once suffices, and the cosmetic preparation is already contaminated with bacteria, fungi and/or yeasts and they multiply.

The build-up of germs in cosmetics is to be avoided.

A preserving effect could be achieved by means of the packaging, although this type of preservation is very expensive. Some manufacturers are moving away from plastic tubes because, after the contents have been squeezed out, they draw in air and thus germs. However, the small opening of a tube is less inviting for germs than the large surface of a pot. Some companies therefore use tubes made of aluminum because, as a result, the drawing-in of air is avoided. More and more frequently, cosmetics are bottled in dispensers because they are particularly hygienic by virtue of their construction.

How susceptible a product is to the build-up of germs, however, also depends on the formulation. Bacteria and yeast fungi require water and a neutral medium in order to reproduce. Massage oils or soaps with a high pH therefore do not have to be preserved. However, in the case of a cream, lotion or a shower gel, if water is right at the top of the list of ingredients, it generally requires a correspondingly powerful preservation.

The most important preserving ingredients in cosmetics were for a long time alcohol and essential oils. However, up to 10% alcohol—depending on water fraction and ingredients—is required to preserve a lotion. However, alcohol is being dispensed with more and more often because it degreases and dries out the skin.

The essential oils of numerous healing plants such as thyme, cloves or rosemary are effective against bacteria. The art consists in combining the oils for each product such that the mixture has a broad spectrum of action. With this type of preservation, the intensive odor of the essential oils is very disadvantageous, the skin also smells very strongly of these herbs and often such natural oils are phytotoxic if one goes into the sun following application.

Preservatives have the task of killing off bacteria, yeasts and fungi. Accordingly, they are aggressive—also to the skin. Many of the artificial preservatives can irritate the skin and lead to redness and a rash. Use for many years increases the risk of the substances sensitizing the organism, leading to allergic reactions.

Known allergens are formaldehyde and substances which release formaldehyde in the product. The organohalogen compounds, to which many preservatives belong, also have a high allergic potential.

Cosmetic preparations have to be formulated with long-term stability to microbial contamination and nevertheless have a very good skin compatibility.

The microbial stability has hitherto been solved by adding preservatives.

Known preservatives are the compounds referred to as parabens, in particular 4-hydroxy-benzoic acid and esters thereof, methylparaben, ethylparaben, propylparaben, isopropylparaben, butylparaben, isobutylparaben, phenylparaben.

Furthermore, phenoxyethanol, ethylene glycol monophenyl ether, phenyl glycol, Phenoxetol®, is known for preserving cosmetic compositions.

However, to provide preparations without preservatives is one wish of consumers.

Further known auxiliaries for microbial stabilization are benzethonium chloride, lauroyl ethyl arginate, octopirox and methylisothiazolinone.

Benzethonium chloride, benzyldimethyl (4-{2-[4-(1,1,3,3-tetramethylbutyl)phenoxy]ethoxy}ethyl)ammonium chloride, of the structure

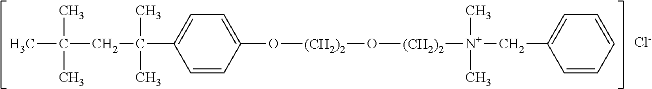

exhibits, like many quaternary ammonium compounds, biocidal properties.

As a disinfectant, it is used in cleaning compositions, but also in medicaments (spermicides, suckable tablets to combat angina). Grapefruit extracts can comprise benzethonium chloride in concentrations of 7-11%.

In cosmetics, the use of benzethonium chloride is known, as described for example in WO 2007015243 A1, WO 2004034964 A1, EP 1310234 A1, WO 2002008377 A1.

Lauryl ethyl arginate, lauroyl ethyl arginate or also referred to as ethyl lauroyl arginate HCl, for example available as Aminat G from Vedeqsa, Inc., is used as a preservative in cosmetic preparations.

Aminat-G (INCI: glycerol and ethyl lauroyl arginate HCl) exhibits a large antimicrobial spectrum and is FDA tested inter alia as additive in foods (see e.g. WO 2008133724 A1).

Piroctone olamine, 1-hydroxy-4-methyl-6-(2,4,4-trimethylpentyl)-2(1H)-pyridone, also referred to as octopirox, is an antimicrobial which is used as preservative and e.g. in hair-washing compositions as an antidandruff substance. Octopirox is highly skin-compatible and physiologically indifferent.

Octopirox has the structure

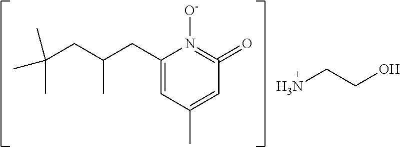

Methylisothiazolinones (2-methyl-2H-isothiazol-3-one (MIT)) are a class of compounds with high microbicidal activity which is used primarily for preserving technical products and only a few cosmetic compositions.

In cosmetic preparations, methylisothiazolinone is described for example in U.S. Pat. No. 6,511,673 or EP 1488699 A1 in combination with parabens or phenoxyethanol, respectively.

Further uses of methylisothiazolinone are disclosed in EP 1525797 A1, EP 1527684 A1, EP 1527685 A1, EP 1621076 A1.

In view of increasing adverse effects on health and allergies and also the pressure by consumers for more ecologically compatible cosmetics, there is the problem that these preparations nevertheless have to have adequate stability and primarily skin compatibility.

Furthermore, a simple replacement or the avoidance of parabens and/or phenoxyethanol without losses in terms of the stability and application properties is not directly possible.

In nature, most bacteria do not occur individually and in freely movable form, but predominantly in biofilms which consist of bacteria growing into microcolonies and the exopolysaccharides formed by them. As a result, the bacteria properties change with influences from the environment. In the literature, greatly increased resistances to antimicrobial active ingredients are described in the case of biofilm-forming bacteria which were tested positive beforehand in liquid culture. Thus, e.g. in medicine, particularly biofilm-forming Staphylococcus epidermidis strains on implants pose large problems. These infections can only be treated with difficulty using conventional antibiotics. A biofilm-dissolving and/or -inhibiting effect in addition to the antimicrobial effectiveness increases the efficiency of a preservative significantly.

It is therefore an object to provide cosmetic or dermatological preparations which have adequate microbiological long-term stability. It is an additional object that these preparations are formulated in a skin-, hair- and application-friendly form and have a low skin irritation potential.

The invention comprises benzethonium chloride, methylisothiazolinone, piroctone olamine and/or lauroyl ethyl arginate in cosmetic or dermatological preparations which comprise no further preservatives, in particular comprise no agents with phenol groups, in particular are free from parabens and/or phenoxyethanol.

The fraction of benzethonium chloride, methylisothiazolinone, piroctone olamine and/or lauroyl ethyl arginate is to be selected preferably in the range from 0.01 to 5% by weight, in particular in the range from 0.1 to 2% by weight, based on the total mass of the preparation.

In the preparation, advantageously two or more, i.e. two, three or all, of the substances benzethonium chloride, methylisothiazolinone, piroctone olamine and/or lauroyl ethyl arginate may be present.

In preparations for cleansing the skin and/or cleansing and/or caring for the hair, preferably benzethonium chloride and/or lauroyl ethyl arginate are present.

It is surprising here that the preparations containing the substances according to the invention, benzethonium chloride, methylisothiazolinone, piroctone olamine and/or lauroyl ethyl arginate, instead of parabens and/or phenoyethanol nevertheless have adequate microbial stability.

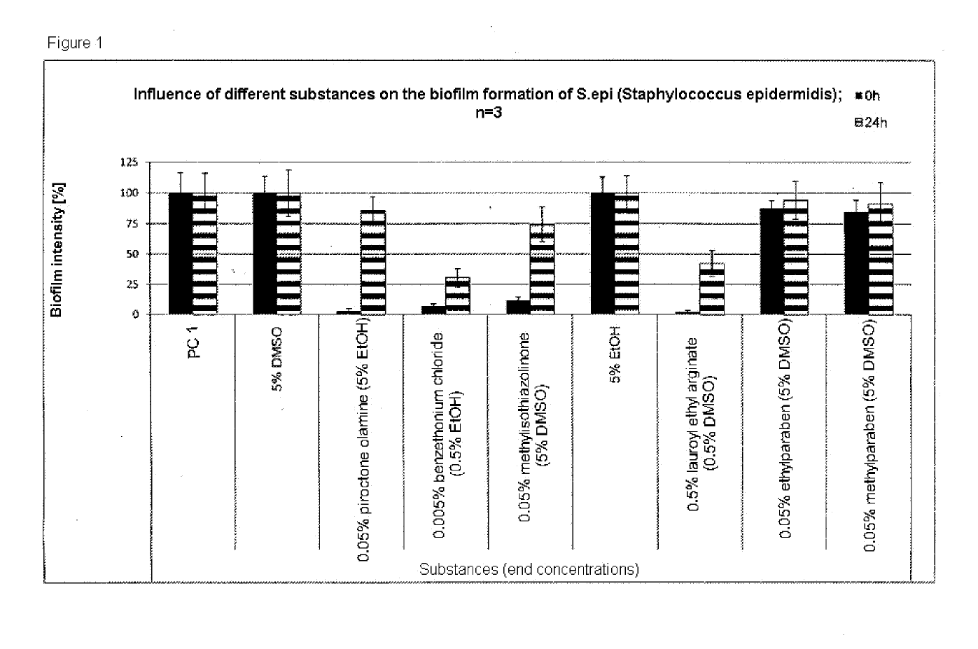

FIG. 1 shows this microbial stability in an impressive manner.

In the course of the biofilm assay, the biofilm inhibition at timepoint 0 h and also the biofilm-dissolving effect at timepoint 24 h is investigated using a color reaction on a microtiter plate scale. The measurement results of the investigated active ingredients are based on the positive control (PC1). Neither ethylparaben nor methylparaben exhibited effectiveness. In contrast to this, benzethonium chloride, methylisothiazolinone, piroctone olamine and lauroyl ethyl arginate exhibited a very good biofilm-inhibiting effectiveness. Moreover, a very good biofilm-dissolving effect could be shown for benzethonium chloride and lauroyl ethyl arginate. With piroctone olamine, methylisothiazolinone as well, a slight biofilm dissolution could be achieved.

According to the invention, benzethonium chloride, methylisothiazolinone, piroctone olamine and/or lauroyl ethyl arginate are to be used in cosmetic or dermatological preparations for improving the biofilm-inhibiting effectiveness.

Preference is given to this use if the addition of parabens or phenoxyethanol is dispensed with.

In the case of the conventional antibacterial active ingredients, a distinction is made between the bacteriostatic effect and the bactericidal effect. In the case of the latter, the bacteria are killed off by the active ingredient. By contrast, the bacteriostatic effect prevents the bacteria from reproducing. The preservatives used in cosmetic formulations for preventing the build-up of germs are generally types of bactericidal active ingredients. However, as a result of using classic antimicrobial actives, firstly the skin flora is adversely affected and, secondly, in recent years resistance developments have increasingly been observed. In contrast to conventional antimicrobial active ingredients, the anti-biofilm active ingredients do not interfere directly in the bacterial metabolism, but only prevent the formation of the biofilm protecting the bacteria, or dissolve said film. A resistance development is therefore ruled out.

The test shown in FIG. 1 was carried out with Staphylococcus epidermidis, a germ which causes little harm for people with a normal immunological defense. Staphylococcus epidermidis (syn. Staphylococcus albus, Micrococcus epidermidis, Staphylococcus saprophyticus) is a Gram-positive, plasmacoagulase-negative, saprophagic bacterium which colonizes the human skin and mucosa. Furthermore, it can be found on foods and also colonizes on polymeric surfaces (e.g. packagings). However, in a hospital, in the case of immunocompromised people, it can, in cases of lack of cleanliness, be a cause of serious diseases.

S. epidermidis has a large spectrum for antibiotic resistances. This is the case primarily against penicillin and methicillin. The proportion of resistant strains is in the meantime 70%.

It is therefore the ideal bacterium for showing the advantageous effect of the cosmetic preparations according to the invention.

The preparations according to the invention are preferably paraben- and/or phenoxyethanol-free and nevertheless have an adequate microbial stability.

Paraben-free or phenoxyethanol-free means that the fraction of parabens and/or phenoxyethanol is less than 1% by weight, based on the total mass of the preparation. Advantageously, the fraction of parabens and phenoxyethanol is 0% by weight.

The cosmetic preparations according to the invention can also comprise cosmetic auxiliaries and further active ingredients as are customarily used in such preparations, e.g. substances for preventing foaming, dyes and colored pigments, thickeners, moisturizing and/or humectant substances, fats, oils, waxes or other customary constituents of a cosmetic or dermatological formulation such as alcohols, polyols, polymers, foam stabilizers, electrolytes, organic solvents or silicone derivatives provided the addition does not adversely affect the required properties with regard to the microbiological stability and also skin and hair compatibility.

The preparations according to the invention can advantageously in the form of aqueous gels, aqueous-alcoholic solutions, O/W or W/O emulsions of soft, semi-soft or solid consistency, microemulsions, W/O/W or O/W/O emulsions. Ideal application forms in this connection are face creams, sera, make-up, foundation, body lotions, body milks, hand cream, deodorant roll-on, deodorant stick, deodorant aerosol, deodorant atomizer.

The use in rinse-off products such as shampoos, shower gels or hand-washing gels are also preferred.

The examples below show cosmetic preparations according to the invention with good microbial stability.

The numerical data are fractions by weight, based on the total mass of the preparation.

W/O Emulsions

| | 1 | 2 | 3 | 4 | 5 |
|---|---|---|---|---|---|
| Triglycerin | 1.0 | 0.5 | 0.25 | 2.0 | 3.0 |
| diisostearate | | | | | |
| Diglycerin | 1.0 | 1.5 | 1.75 | 3.0 | 2.0 |
| dipolyhydroxystearate | | | | | |
| Paraffin oil | 12.5 | 10.0 | 8.0 | 5.0 | 11.5 |
| Petrolatum | 8.0 | 6.0 | 5.0 | 12.0 | 2.5 |
| Hydrogenated | 2.0 | 1.0 | 2.5 | 5.0 | 0.25 |
| cocoglycerides | | | | | |
| Decyloleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Silicone elastomer gel | 7.5 | 12.5 | 2.5 | 5 | 20 |
| Magnesium sulfate | 0.5 | 0.6 | 0.5 | 0.7 | 1.0 |
| Glycerin | 3.0 | 5.0 | 10.0 | 15.0 | 1.5 |
| Citric acid | 0.2 | 0.1 | 0.2 | 0.3 | 1.0 |
| Sodium citrate | 0.2 | 0.05 | 0.4 | 0.3 | 2.0 |
| Perfume | q.s. | q.s. | q.s. | q.s. | q.s. |
| Ethanol | 2.0 | — | 5.0 | — | — |
| Caprylic-/capric | 2.0 | 2.5 | 3. | 5.0 | 0.5 |
| triglycerides | | | | | |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Water | ad 100 | ad 100 | ad 100 | ad 100 | ad 100 |

W/O Emulsions

| | 6 | 7 | 8 | 9 | 10 |
|---|---|---|---|---|---|
| PEG-30 | — | 0.5 | 0.25 | — | 3.0 |
| Dipolyhydroxystearate | | | | | |
| Lanolin alcohol | 1.0 | 1.5 | 1.75 | 3.0 | — |
| Paraffin oil | 12.5 | 10.0 | 8.0 | 5.0 | 10.5 |
| Petrolatum | 8.0 | 6.0 | 5.0 | 12.0 | 2.5 |
| Hydrogenated | 2.0 | 1.0 | 2.5 | 5.0 | 0.25 |
| cocoglycerides | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Microcrystalline | 0.5 | 1.0 | 0.75 | 0.25 | 0.1 |
| cellulose | | | | | |
| Magnesium sulfate | 0.5 | 0.6 | 0.5 | 0.7 | 1.0 |
| Glycerin | 3.0 | 5.0 | 10.0 | 15.0 | 1.5 |
| Citric acid | 0.2 | 0.1 | 0.2 | 0.3 | 1.0 |
| Perfume | q.s. | q.s. | q.s. | q.s. | q.s. |
| 1,3-Butylene glycol | 2.0 | — | 5.0 | — | — |
| Caprylic/capric | 2.0 | 2.5 | 3.0 | 5.0 | 0.5 |
| triglycerides | | | | | |
| Silicone elastomer gel | 5.0 | — | — | 3.0 | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Talc | — | — | 0.05 | — | 0.1 |
| Water | ad 100 | ad 100 | ad 100 | ad 100 | ad 100 |

W/S Emulsion

| | 11 | 12 | 13 | 14 | 15 |
|---|---|---|---|---|---|
| Cetyl PEG/PPG-10/1 | 1.0 | — | — | 3.0 | 5.0 |
| dimethicone | | | | | |
| Cylomethicone + | 10.0 | 12.5 | 25 | — | — |
| PEG/PPG-18/18 | | | | | |
| Dimethicone (90:10) | | | | | |
| Cyclic silicone oil | 12.5 | 15 | 22.0 | 20.0 | 15.5 |
| (Cyclomethicone) | | | | | |
| Linear silicone oil | 5.0 | 13.0 | 5.0 | 12.0 | 15.0 |
| (Dimethicone) | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerin | 3.0 | 5.0 | 10.0 | 15.0 | 1.5 |
| Citric acid | 0.2 | 0.1 | 0.2 | 0.3 | 1.0 |
| Sodium citrate | 1.0 | 0.1 | 0.4 | 0.9 | 2.5 |
| Perfume | q.s. | q.s. | q.s. | q.s. | q.s. |
| Microcrystalline | 1.0 | 0.1 | 0.5 | 0.25 | 0.1 |
| cellulose | | | | | |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Modified starch | — | 2.5 | — | 0.15 | — |
| Water | ad 100 | ad 100 | ad 100 | ad 100 | ad 100 |

W/S Emulsions

| | 16 | 17 | 18 | 19 | 20 |
|---|---|---|---|---|---|
| Cetyl PEG/PPG-10/1 | 1.0 | — | — | 3.0 | 5.0 |
| Dimethicone | | | | | |
| Cylomethicone + | 10.0 | 12.5 | 25 | — | — |
| PEG/PPG-18/18 | | | | | |
| Dimethicone (90:10) | | | | | |
| Cyclic silicone oil | 12.5 | 15 | 8.0 | 20.0 | 17.5 |
| (Cyclomethicone) | | | | | |
| Linear silicone oil | 5.0 | 13.0 | 5.0 | 12.0 | 15.0 |
| (Dimethicone) | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerin | 3.0 | 5.0 | 10.0 | 15.0 | 1.5 |
| Lactic acid | 0.2 | 0.1 | 0.2 | — | — |
| Sodium lactate | 0.2 | 1.0 | 0.05 | — | — |
| Perfume | q.s. | q.s. | q.s. | q.s. | q.s. |
| Microcrystalline | 1.0 | 0.1 | 1.5 | 2.5 | 0.1 |
| cellulose | | | | | |
| Stearyl dimethicone | 0.5 | — | 0.7 | — | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Modified starch | — | 2.5 | — | 0.15 | — |
| Water | ad 100 | ad 100 | ad 100 | ad 100 | ad 100 |

Silicone-in-Water Emulsion

| | 21 | 22 | 23 | 24 | 25 |
|---|---|---|---|---|---|
| Bis-PEG/PPG-16/16 | 1.0 | 2.0 | 8.0 | 3.0 | 5.0 |
| PEG/PPG16/16 | | | | | |
| Dimethicone, | | | | | |
| Caprylic/capric | | | | | |
| triglyceride | | | | | |
| Cyclic silicone oil | 12.5 | 15 | 25.0 | 10.0 | 7.5 |
| (Cyclomethicone) | | | | | |
| Linear silicone oil | 5.0 | 15.0 | 5.0 | 12.0 | 15.0 |
| (Dimethicone) | | | | | |
| Octyldodecanol | 0.5 | 1.0 | 0.75 | 3.0 | 0.25 |
| Glycerin | 5.0 | 7.5 | 10.0 | 3.0 | 1.0 |
| Panthenol | 0.5 | 1.0 | 0.75 | 0.25 | 0.1 |
| Perfume | q.s. | q.s. | q.s. | q.s. | q.s. |
| Stearyl dimethicone | 0.5 | — | 0.7 | — | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Modified starch | — | 2.5 | — | 0.15 | — |
| Water | ad 100 | ad 100 | ad 100 | ad 100 | ad 100 |

O/W Emulsion

| | 26 | 27 | 28 | 29 | 30 |
|---|---|---|---|---|---|
| Glyceryl stearate | 1.0 | 1.5 | 1.5 | 0.5 | 0.25 |
| citrate | | | | | |
| Cetyl stearylalcohol | 2.5 | 5 | 0.5 | 2.0 | 1.5 |
| Ethylhexyl | 5.0 | 3.0 | 2.0 | 2.0 | 5.0 |
| methoxycinnamate | | | | | |
| Ethylhexylglycerin | 1 | — | 0.5 | 1 | — |
| Aluminium | — | 1 | 0.02 | 1 | — |
| chlorohydrate | | | | | |
| Ethanol | — | — | 3.0 | 1 | — |
| Ammonium Acryloyl- | 0.5 | 0.75 | 0.5 | 2.0 | 0.25 |
| dimethyltairate/VP | | | | | |
| Copolymer | | | | | |
| Blue water-soluble | 0.5 | 1.0 | 0.4 | 3.0 | 0.25 |
| dye (Blue No. 1) | | | | | |
| Carbomer | — | — | 0.2 | — | 0.1 |
| Phosphated starch | 1.0 | — | 4.0 | — | 0.5 |
| 8-Hexadecene-1,16-di- | 0.5 | 0.75 | 1.0 | 1.5 | 2.5 |
| carboxylic acid | | | | | |
| Octyldodecanol | 0.5 | — | 1.0 | — | 2.0 |
| Caprylic/capric | — | — | 1.0 | — | 0.5 |
| triglyceride | | | | | |
| Myristyl myristate | — | 1.5 | 1.0 | 2.5 | — |
| Perfume | q.s. | q.s. | q.s. | q.s. | q.s. |
| Sea salt | — | — | 0.01 | 0.5 | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Glycerin | 5 | 10 | 7.5 | 15 | 7.5 |
| Tocopherol acetate | 0.5 | 0.25 | 0.5 | 0.1 | — |
| Titanium dioxide | — | 2.5 | 1.2 | 0.15 | — |
| Water | ad 100 | ad 100 | ad 100 | ad 100 | ad 100 |

O/W Emulsion

| | 31 | 32 | 33 | 34 | 35 |
|---|---|---|---|---|---|
| Polyethylene- | 1 | — | 2.5 | 2 | 1.5 |
| glycol (21)stearyl- | | | | | |
| ether | | | | | |
| Polyethylene- | 1 | — | 5.5 | 3 | 7.5 |
| glycol(2) stearyl- | | | | | |
| ether | | | | | |
| Cetearyl glucoside | — | 8 | — | — | — |
| Cyclomethicone | 12.5 | 15 | 28.0 | 25.0 | 17.5 |
| Dimethicone | 5.0 | 13.0 | 5.0 | 12.0 | 15.0 |
| Behenyl alcohol | 3 | 2 | — | 1 | — |
| Stearyl alcohol | 3 | 2 | — | 2 | — |
| Cetyl stearyl alcohol | 3 | 4 | — | — | 2 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Glycerin | 5 | 10 | 15 | 3 | 7.5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Panthenol | 0.5 | 1.0 | 0.75 | 0.25 | 0.1 |
| Perfume | q.s. | q.s. | q.s. | q.s. | q.s. |
| Modified starch | 0.5 | — | — | 0.15 | — |
| Water | ad 100 | ad 100 | ad 100 | ad 100 | ad 100 |

O/W Emulsion

| | 36 | 37 | 38 | 39 | 40 |
|---|---|---|---|---|---|
| Glyceryl stearate | 1.0 | 0.5 | 0.1 | 0.5 | 0.3 |
| citrate | | | | | |
| Polyethylene- | 10.0 | 1.0 | 5 | — | — |
| glycol(20) cetearyl- | | | | | |
| ether | | | | | |
| Triglycerin | — | — | — | — | 2.5 |
| methylglucose | | | | | |
| distearate | | | | | |
| Cyclomethicone | — | — | — | 1 | — |
| Dimethicone | 0.5 | 3.0 | 0.75 | 1.5 | 0.2 |
| Behenyl alcohol | 1 | — | 2 | 1 | 0.2 |
| Dicaprylyl carbonate | 3 | 5 | 10 | 15 | 5 |
| Stearyl alcohol | — | — | — | 1 | 0.2 |
| Cetyl stearyl alcohol | — | — | 1 | 1 | 0.2 |
| Tocopherol | 0.5 | 0.5 | 0.75 | 0.25 | 0.1 |
| Octyldodecanol | 0.5 | — | 0.75 | 3.0 | 0.25 |
| Panthenol | 0.5 | — | 0.75 | 0.25 | 0.1 |
| Carbomer | 0.05 | 0.35 | 0.15 | 0.1 | — |
| Perfume | q.s. | q.s. | q.s. | q.s. | q.s. |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Sorbitol | 10 | — | — | 5 | — |
| Butylene glycol | — | — | — | 5 | 10 |
| Propylene glycol | — | — | 10 | 5 | — |
| Glycerin | — | 7.5 | — | — | — |
| Water | ad 100 | ad 100 | ad 100 | ad 100 | ad 100 |

O/W Emulsion

| | 41 | 42 | 43 | 44 | 45 |
|---|---|---|---|---|---|
| Glyceryl stearate | 1.0 | 0.75 | 0.1 | 0. | 0.3 |
| citrate | | | | | |
| Polyethylene- | 1.0 | — | 2 | 1.5 | — |
| glycol(20) cetearyl- | | | | | |
| ether | | | | | |
| Triglycerin | — | 5.5 | — | — | 2.5 |
| methylglucose | | | | | |
| distearate | | | | | |
| Ethylbutylacetyl | 5 | 10 | 15 | 20 | 7.5 |
| aminopropionate | | | | | |
| Cyclomethicone | 2 | 4 | 6 | 1 | 3 |
| Dimethicone | — | 0.5 | 0.75 | — | — |
| Behenyl alcohol | 1 | — | 2 | 1 | 0.2 |
| Stearyl alcohol | — | 1 | — | 1 | 0.2 |
| Cetyl stearyl alcohol | — | — | 1 | 1 | 0.2 |
| Medicinal white oil | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Carbomer | 0.05 | 0.1 | 0.15 | 0.1 | — |
| Perfume | q.s. | q.s. | q.s. | q.s. | q.s. |
| Dicaprylyl carbonate | 3 | 5 | 10 | 15 | 5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Tocopherol | 0.5 | 1.0 | 0.75 | 0.25 | 0.1 |
| Caprylic/capric | 1 | 2 | 3 | 5 | 10 |
| triglyceride | | | | | |
| Modified starch | — | 2.5 | — | 0.15 | — |
| Glycerin | 3 | 5 | 8 | 12 | 10 |
| Water | ad 100 | ad 100 | ad 100 | ad 100 | ad 100 |

O/W Emulsion:

| | 46 | 47 | 48 | 49 | 50 |
|---|---|---|---|---|---|
| Panthenol | 1.5 | 0 | 0 | 0.7 | 0 |
| Butylene glycol | — | — | — | 5 | 10 |
| Propylene glycol | — | — | 10 | 5 | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Medicinal white oil | 0 | 3 | 3 | 0 | 0 |
| Isopropyl palmitate | 1 | 0 | 0 | 0 | 0 |
| Caprylic/capric | 0 | 3 | 3 | 0 | 1.00 |
| triglyceride | | | | | |
| Cetearyl alcohol | 0 | 0 | 0 | 2.5 | 4.00 |
| Cetyl alcohol | 0 | 3 | 3 | 0 | 0 |
| Linear silicone oil | 0 | 3 | 3 | 0 | 0.50 |
| Cyclic silicone oil | 0 | 3 | 3 | 0 | 0 |
| C12-15 Alkyl benzoate | 0 | 0 | 0 | 0 | 2.00 |
| Shea butter | 0 | 0.5 | 0.5 | 0 | 0 |
| Dicaprylyl ether | 0 | 0 | 0 | 10 | 0 |
| Dicaprylyl carbonat | 2 | 0 | 0 | 0 | 0 |
| Glyceryl stearate | 0 | 1.2 | 1.2 | 2.4 | 2.4 |
| Tapioca starch | 1 | 0 | 0 | 0 | 0 |
| Glycerin | 5 | 8 | 10 | 8 | 8 |
| Citric acid | 0.0050 | 0 | 0 | 0 | 0 |
| Aqueous sodium | 0.35 | 0.01 | 0.01 | 0.25 | 1 |
| hydroxide 45% | | | | | |
| Polyglyceryl-10 | 0 | 1.8 | 0 | 0 | 0 |
| myristate | | | | | |
| Polyglyceryl-10 | 0.7 | 0 | 1.8 | 1 | 1 |
| stearate | | | | | |
| Polyacrylic acid, | 0.5 | 0.02 | 0.02 | 0 | 0 |
| Na salt | | | | | |
| Acrylic Acid/VP | 0 | 0 | 0 | 0.5 | 0.75 |
| Crosspolymer | | | | | |
| Trisodium EDTA | 0 | 1 | 1 | | 1 |
| Ethylhexyl | 0 | 0 | 0 | 2 | 0 |
| methoxycinnamate | | | | | |
| Butylmethoxy | 0 | 0 | 0 | 0 | 2 |
| dibenzoylmethane | | | | | |
| Phenylbenzimidazole | 0 | 0 | 0 | 0 | 2 |
| sulfonic acid | | | | | |
| Ethylhexyl salicylate | 0 | 0 | 0 | 0 | 2 |
| Titanium dioxide + | 0 | 0 | 0 | 0.3600 | 0 |
| Trimethoxycaprylyl | | | | | |
| silane | | | | | |
| Octocrylene | 0 | 0 | 0 | 0 | 2 |
| Perfume | 0.10 | 0.35 | 0.35 | 0 | 0.20 |
| Water | ad 100 | ad 100 | ad 100 | ad 100 | ad 100 |

Shower Gels

| | 51 | 52 | 53 | 54 | 55 | 56 |
|---|---|---|---|---|---|---|
| Sodium laurylether sulfate | 7 | 6 | 5.5 | 7 | 6 | 5 |
| Cocamidopropyl betaine | 4 | — | 5.5 | — | 5 | — |
| Sodium cocoamphoacetate | — | 4.5 | — | 5 | — | 4 |
| Sodium cocoylglutamate | 1 | — | — | — | — | 1.5 |
| PEG-7 Glyceryl cocoate | — | 3 | — | — | 2 | — |
| PEG-9 Cocoglycerides | 1.5 | — | — | — | — | 2 |
| PEG-10 Olive glycerides | — | — | 2.5 | 1.5 | — | — |
| Polyquaternium 10 | — | 0.2 | — | 0.2 | — | — |
| Polyquaternium 7 | — | — | — | — | 0.4 | — |
| Guar Hydroxypropyltrimonium | — | — | — | — | — | — |
| Chloride | | | | | | |
| Ethyleneglycol distearate | — | — | 1.2 | — | — | 1 |
| PEG-3 Distearate | 1 | — | — | — | — | — |
| Styrene Acrylate Copolymer | — | — | — | — | 0.7 | — |
| PEG-200 Hydrogenated glyceryl | 1 | 0.8 | 1 | 0.5 | 1 | 0.8 |
| palmitate | | | | | | |
| PEG-40 Hydrogenated castor oil | 0.4 | 0.3 | 0.4 | 0.25 | 0.4 | 0.3 |
| Sodium chloride | 0.2 | 0.2 | 0.3 | 0.1 | 0.1 | 0.2 |
| Ethyl lauroylarginate HCl | 0.4 | 0.4 | 0.4 | 0.4 | 0.4 | 0.4 |
| Benzethonium chloride | 0.05 | 0.05 | 0.05 | 0.05 | 0.05 | 0.05 |
| Perfume | q.s. | q.s. | q.s. | q.s. | q.s. | q.s. |
| Water | ad 100 | ad 100 | ad 100 | ad 100 | ad 100 | ad 100 |
| | 57 | 58 | 59 | 60 | 61 | 62 |
| Sodium laurylether sulfate | 7 | 5.5 | 5.5 | 7 | 6 | 5 |
| Cocamidopropyl betaine | 4 | — | 5.5 | — | 5 | — |
| Sodium cocoamphoacetate | — | 4.5 | — | 5 | — | 4.5 |
| Sodium cocoylglutamate | — | 1.0 | — | 1 | 1 | — |
| PEG-7 Glyceryl cocoate | — | 2 | 2 | — | 2 | — |
| PEG-9 Cocoglycerides | 1.5 | — | — | — | — | 2 |
| PEG-10 Oliveglycerides | — | — | — | 1.5 | — | — |
| Polyquaternium 10 | — | 0.2 | — | 0.2 | — | — |
| Polyquaternium 7 | — | — | — | — | — | — |
| Guar Hydroxypropyltrimonium | — | — | 0.2 | — | — | — |
| Chloride | | | | | | |
| Ethyleneglycol distearate | 1 | — | 1.2 | — | — | — |
| PEG-3 Distearate | — | — | — | — | — | 1 |
| Styrene Acrylate Copolymer | — | — | — | 0.6 | — | — |
| PEG-200 Hydrogenated glyceryl | 1 | 0.8 | 0.8 | 0.5 | 1 | 0.8 |
| palmitate | | | | | | |
| PEG-40 Hydrogenated castor oil | 0.4 | 0.3 | 0.4 | 0.25 | 0.4 | 0.3 |
| Sodium chloride | 0.2 | 0.2 | 0.3 | 0.1 | 0.1 | 0.2 |
| Ethyl lauroyl arginate HCl | 0.4 | 0.4 | 0.4 | 0.4 | 0.4 | 0.4 |
| Benzethonium chloride | 0.05 | 0.05 | 0.05 | 0.05 | 0.05 | 0.05 |
| Perfume | q.s. | q.s. | q.s. | q.s. | q.s. | q.s. |
| Water | ad 100 | ad 100 | ad 100 | ad 100 | ad 100 | ad 100 |
| | 63 | 64 | 65 | 66 | 67 | 68 |
| Sodium laurylether sulfate | 6 | 5.5 | 5.5 | 7 | 7 | 6.5 |
| Cocamidopropyl betaine | 5 | — | 5.5 | — | 4 | 5 |
| Sodium cocoamphoacetate | — | 5.5 | — | 5 | — | — |
| Sodiium cocoylglutamate | 1 | — | — | 1 | — | — |
| PEG-7 Glycerylcocoate | 2 | 2 | 2 | — | — | 2 |
| PEG-9 Cocoglycerides | — | — | — | — | 1.5 | — |
| PEG-10 Oliveglycerides | — | — | — | 1.5 | — | — |
| Polyquaternium 10 | — | 0.2 | — | 0.2 | — | — |
| Polyquaternium 7 | — | — | 0.4 | — | — | 0.3 |
| Guar Hydroxypropyltrimonium | — | — | — | — | — | — |
| Chloride | | | | | | |
| Ethyleneglycol distearate | — | — | 1.2 | — | 1 | — |
| PEG-3 Distearate | — | — | — | — | — | — |
| Styrene Acrylate Copolymer | — | 0.8 | — | — | — | 0.7 |
| PEG-200 Hydrogenated glyceryl | 1 | 0.8 | 0.8 | 0.5 | 1 | 0.8 |
| palmitate | | | | | | |
| PEG-40 Hydrogenated castor oil | 0.4 | 0.3 | 0.4 | 0.25 | 0.4 | 0.3 |
| Sodium chloride | 0.1 | 0.2 | 0.3 | 0.1 | 0.2 | 0.2 |
| Ethyl lauroyl arginate HCl | 0.4 | 0.4 | 0.4 | 0.4 | 0.4 | 0.4 |
| Benzethonium chloride | 0.05 | 0.05 | 0.05 | 0.05 | 0.05 | 0.05 |
| Perfume | q.s. | q.s. | q.s. | q.s. | q.s. | q.s. |
| Water | ad 100 | ad 100 | ad 100 | ad 100 | ad 100 | ad 100 |
| | 69 | 70 | 71 | 72 | 73 | 74 |
| Sodium laurylether sulfate | 6 | 5.5 | 7 | 6 | 6.5 | 5.5 |
| Cocamidopropyl betaine | 5 | 5.5 | — | 5 | 5 | — |
| Sodium cocoamphoacetate | — | — | 5 | — | — | 5.5 |
| Sodium cocoylglutamates | — | — | 1 | — | — | — |
| PEG-7 Glyceryl cocoate | 2 | 2 | — | 2 | 2 | 2 |
| PEG-9 Cocoglycerides | — | — | — | — | — | — |
| PEG-10 Oliveglycerides | — | — | 1.5 | — | — | — |
| Polyquaternium 10 | — | — | 0.2 | — | — | 0.2 |
| Polyquaternium 7 | 0.4 | — | — | 0.4 | — | — |
| Guar Hydroxypropyltrimonium | — | — | 0.1 | 0.2 | 0.2 | — |
| Chloride | | | | | | |
| Ethyleneglycol distearate | — | 1.2 | — | — | — | — |
| PEG-3 Distearate | 0.7 | — | — | — | — | 0.8 |
| Styrene Acrylate Copolymer | — | — | — | 0.7 | 0.7 | — |
| PEG-200 Hydrogenated glyceryl | 1 | 0.8 | 0.5 | 1 | 0.8 | 0.8 |
| palmitate | | | | | | |
| PEG-40 Hydrogenated castor oil | 0.4 | 0.4 | 0.25 | 0.4 | 0.3 | 0.3 |
| Sodium chloride | 0.1 | 0.3 | 0.1 | 0.1 | 0.2 | 0.2 |
| Ethyl lauroyl arginate HCl | 0.4 | 0.4 | 0.4 | 0.4 | 0.4 | 0.4 |
| Benzethonium chloride | 0.05 | 0.05 | 0.05 | 0.05 | 0.05 | 0.05 |
| Perfume | q.s. | q.s. | q.s. | q.s. | q.s. | q.s. |
| Wasser | ad 100 | ad 100 | ad 100 | ad 100 | ad 100 | ad 100 |

Shampoos

| | 75 | 76 | 77 | 78 | 79 | 80 | 81 | 82 |
|---|---|---|---|---|---|---|---|---|
| Water | Ad 100 | Ad 100 | Ad 100 | Ad 100 | Ad 100 | Ad 100 | Ad 100 | Ad 100 |
| Sodium laureth sulfate | 10 | 10 | 9 | 12 | 10 | 12 | 12 | 10 |
| Cocamidopropyl betaine | 2 | 1.5 | 3 | 1.5 | 3 | 2 | 2 | 3 |
| Cocamide DEA | 3 | 2 | 2 | 3 | | | | |
| Polyquaternium-10 | 0.2 | | 0.2 | | 0.3 | 0.3 | | |
| Polyquaternium-7 | | | 0.3 | 0.3 | 0.2 | | 0.3 | |
| Guar Hydroxypropyltrimonium | 0.2 | 0.3 | | 0.1 | | 0.1 | 0.1 | 0.3 |
| Chloride | | | | | | | | |
| Dimethicone | 2 | 1 | 2 | 2.5 | | | | |
| Perfume | 0-1 | 0-1 | 0-1 | 0-1 | 0-1 | 0-1 | 0-1 | 0-1 |
| pH Adjuster | q.s. | q.s. | q.s. | q.s. | q.s. | q.s. | q.s. | q.s. |
| PEG-3 Distearate | 1.5 | 1.5 | 1.5 | 1.5 | 1.5 | 1.5 | 1.5 | 1.5 |
| Poly(2-propenal, | 0.01 | 0.03 | 0.10 | 0.25 | 0.50 | 1.00 | 0.03 | 2.00 |
| 2-propenoic acid) | | | | | | | | |
| Piroctone olamine | | | | 0.10 | | | | |
| Ethyl lauroyl arginate HCl | 0.4 | 0.4 | 0.4 | 0.4 | 0.4 | 0.4 | 0.4 | 0.4 |
| Benzethonium chloride | 0.05 | 0.05 | 0.05 | 0.05 | 0.05 | 0.05 | 0.05 | 0.05 |

Conditioner

| | 83 | 84 | 85 | 86 | 87 | 88 |
|---|---|---|---|---|---|---|
| Ethyl lauroyl arginate HCl | 0.4 | 0.4 | 0.4 | 0.4 | 0.4 | 0.4 |
| Benzethonium chloride | 0.05 | 0.05 | 0.05 | 0.05 | 0.05 | 0.05 |
| 1,2-Pentadiol | | | 0.3 | | | |
| 1,2-Hexanediol | | | | 0.4 | | |
| Ethylhexylglycrol | | 0.5 | | | | |
| Priroctone olamine | 0.05 | | | | | |
| Poly(2-propenal, | 0.01 | 0.03 | 0.50 | 0.75 | 1.00 | 2.00 |
| 2-propenoic acid) | | | | | | |
| Water | Ad 100 | Ad 100 | Ad 100 | Ad 100 | Ad 100 | Ad 100 |
| Citric acid | | 0.01 | 0.01 | | | |
| Cetearyl alcohol | | 3.5 | 4.44 | | | |
| Cetyl alcohol | 2.5 | | | 2.5 | 2.5 | 2.5 |
| Trisodium EDTA | | | | | | 1 |
| Lactic acid | 0.8 | | | 0.8 | 0.8 | 0.84 |
| Glycerol | | | 5 | | | |
| Sodium hydroxide | | 0.03 | 0.03 | | | |
| Sodium chloride | 0.01 | | | 0.01 | 0.01 | 0.01 |
| Stearyl alcohol | 4.8 | | | 4.8 | 4.8 | 4.8 |
| Cetrimonium chloride | | 2 | | | | |
| Distearoylethyl | | | 2.86 | | | |
| hydroxyethylmonium | | | | | | |
| methosulfate | | | | | | |
| Oryzanol | 0.05 | | 0.05 | 0.05 | 0.05 | 0.05 |
| Palmitamidopropyl- | | | 1.66 | | | |
| trimonium chloride | | | | | | |
| Coco betaine | 0.85 | | | 0.85 | 0.85 | 0.85 |
| Stearamidopropyl | 2 | | | 1.5 | 2 | 2.5 |
| dimethylamine | | | | | | |
| Water + Silicone Quaternium- | | | 5 | 3 | 2 | 2.5 |
| 18 + Trideceth-6 + | | | | | | |
| Trideceth-12 | | | | | | |
| Behentrimonium methosulfate | | 2.73 | | | | |
| Dimethicone + | 2 | 4 | 1 | 1 | 3 | |
| Cocamidopropyl betaine + | | | | | | |
| C12-15 Pareth-3 + Guar | | | | | | |
| hydroxypropyltrimonium | | | | | | |
| chloride | | | | | | |

| | 89 | 90 | 91 | 92 | 93 | 94 | 95 | 96 |
|---|---|---|---|---|---|---|---|---|
| Ethyl lauroyl arginate HCl | 0.4 | 0.4 | 0.4 | 0.4 | 0.4 | 0.4 | 0.4 | 0.4 |
| Benzethonium Chloride | 0.05 | 0.05 | 0.05 | 0.05 | 0.05 | 0.05 | 0.05 | 0.05 |
| 1,2-Pentadiol | | | 0.5 | | | | | |
| 1,2-Hexanediol | | | | 0.1 | | | | |
| Ethylhexyl glycrol | | 0.5 | | | | | | |
| Glyceryl stearate | | | | | 1 | | | |
| Aqua | Ad 100 | Ad 100 | Ad 100 | Ad 100 | Ad 100 | Ad 100 | Ad 100 | Ad 100 |
| Octyldodecanol | | | | | | 1 | | |
| Citric acid | 0.01 | 0.005 | | | 0.025 | | 0.075 | |
| Cetearyl alcohol | 3.1 | | 5.24 | 5.74 | 1 | | | |
| Cetyl alcohol | | 2 | | | | 3.81 | 4.05 | 4.05 |
| Dimethicone | | 1 | | 1 | | 3 | 3 | 3 |
| Lactic acid | | | | | | 0.85 | 0.85 | 0.85 |
| Benzophenone-4 | | 0.25 | 0.25 | 0.25 | | | | |
| Bis-diglyceryl | 2 | 1 | 1 | 1 | | | | |
| polyacyladipate-2 | | | | | | | | |
| Glycerol | 8.7 | 0 | 0 | 5 | | | | |
| Poly(2-propenal, | 0.01 | 0.03 | 0.10 | 0.25 | 0.50 | 0.75 | 0.03 | 2.00 |
| 2-propenoic acid) | | | | | | | | |
| Water + sodium hydroxide | 0.025 | 0.06 | 0.15 | 0.15 | 0.025 | | | |
| Hydroxyethylcellulose | | | | | 0.75 | 0.25 | 0.25 | 0.25 |
| Sodium chloride | | | | | | 0.01 | 0.01 | 0.01 |
| Hydroxypropyl | 0.25 | | | | | | 0.1 | 0.2 |
| methylcellulose | | | | | | | | |
| Cetearyl alcohol + | 0.2 | | | | | | | |
| behentrimonium chloride | | | | | | | | |
| Stearyl alcohol | | 4 | | | | 7.3152 | 7.776 | 7.776 |
| Cetrimonium chloride | 4 | | | | 0.4 | | | |
| Dicaprylyl ether | | | | | | 1 | | |
| Distearoylethyl | | 2 | 2 | 2.86 | | | | |
| hydroxyethylmonium | | | | | | | | |
| methosulfate + cetearyl | | | | | | | | |
| alcohol | | | | | | | | |
| Oryzanol | | | | | | 0.05 | 0.05 | 0.05 |
| DMDM Hydantoin | | | | | 0.4 | | | |
| Silicone Quaternium-16 + | | | | | 2 | | | |
| Undeceth-11 + | | | | | | | | |
| Butyloctanol + Undeceth-5 | | | | | | | | |
| Cyclomethicone | | 2 | | | | | | |
| Palmitamidopropyltrimonium | | 3 | 1.65 | | | | | |
| chloride + Propylene | | | | | | | | |
| glycol | | | | | | | | |
| Coco betaine | | | | | | 0.85 | 3.22 | 3.22 |
| Polyquaternium-68 | | | | | | 7.5 | 3.75 | 1.875 |
| Stearamidopropyl | | | | | | 2.2 | 2.2 | 2.2 |
| dimethylamine | | | | | | | | |
| Perfume | 0.6 | 0.7 | 0.7 | 0.7 | 0.2 | 0.7 | 0.7 | 0.7 |
| Polyquaternium-89 | 1.5 | 1 | 1.2 | 1.5 | 1 | 2 | 1.5 | 1 |

Skin Cleansing

| | 971 | 98 | 99 | 100 | 101 | 102 | 103 | 104 | 105 | 106 |
|---|---|---|---|---|---|---|---|---|---|---|
| Sodium laureth sulfate | 6.80 | 6.30 | | 8.00 | 8.50 | | 5.40 | | | 1.80 |
| Sodium myreth sulfate | | | 7.10 | | | 4.80 | | 3.20 | 2.70 | |
| Cocoamidopropyl betaine | 5.20 | 4.70 | | 3.00 | 2.80 | 5.10 | 4.80 | 6.40 | 3.70 | |
| Sodium cocoamphoacetate | | | 3.50 | | | | | | | |
| Decyl glucoside | | | 2.30 | 1.80 | 2.00 | 2.80 | | 2.10 | | 0.30 |
| Lauryl glucoside | | | | | | | | | 0.60 | |
| Sodium methyl cocoyl taurate | | | | | | | | | | 0.48 |
| Disodium cocoyl glutamate | | 0.50 | | | | | | | | |
| Acrylates Copolymer | | 2.40 | | | | | | | 2.20 | |
| PEG-200 Hydrogenated | 0.40 | | 2.70 | 0.40 | 0.40 | 1.00 | | | 0.50 | |
| glyceryl palmate | | | | | | | | | | |
| PEG-90 Glyceryl isostearate | | | | 0.14 | 0.30 | | | 0.70 | | |
| PEG-120 Methylglucose | | | | | | | 0.10 | | | |
| dioleate | | | | | | | | | | |
| Laureth-2 | | | | 0.01 | 0.03 | | | 0.07 | | |
| Laureth-4 | 0.30 | | | | | | | | | |
| PEG-40 Hydrogenated | 0.40 | 0.80 | 0.30 | 0.40 | 0.50 | 1.00 | 0.40 | 0.50 | 0.50 | |
| castor oil | | | | | | | | | | |
| PEG-7 Glyceryl cocoate | 2.00 | 1.75 | 0.30 | 0.18 | 0.20 | | 1.00 | | | 0.50 |
| Glycol distearate | 0.60 | | | | | 0.60 | 0.30 | | | |
| Styrene/Acrylate Copolymer | | 0.40 | | | | | | | | |
| Glycerol | 0.30 | | 8.60 | | | 0.30 | | | | 1.70 |
| Sorbitol | | | 17.50 | | | | | | | |
| Polyquaternium-7 | 0.30 | | | | | | | | | |
| Polyquaternium-10 | | | 0.20 | | | | | 0.10 | 0.10 | |
| Sodium chloride | 0.15 | | 0.40 | 0.80 | 0.60 | | | | | |
| Acrylates/C10-30 Alkyl | | | | | | | | 0.90 | | |
| Acrylate Crosspolymer | | | | | | | | | | |
| Xanthan gum | | | | | | | | | | 0.25 |
| Carbomer | | | | | | | | | | 1.20 |
| Polyethylene | | 0.40 | | | | | | | | |
| Poly(2-propenal, | 0.01 | 0.03 | 0.10 | 0.25 | 0.50 | 0.01 | 0.03 | 2.00 | 0.75 | 1.00 |
| 2-propenoic acid) | | | | | | | | | | |
| Ethyl lauroyl arginate HCl | 0.4 | 0.4 | 0.4 | 0.4 | 0.4 | 0.4 | 0.4 | 0.4 | 0.4 | 0.4 |
| Benzethonium chloride | 0.05 | 0.05 | 0.05 | 0.05 | 0.05 | 0.05 | 0.05 | 0.05 | 0.05 | 0.05 |
| Perfume | 0-1 | 0-1 | 0-1 | 0-1 | 0-1 | 0-1 | 0-1 | 0-1 | 0-1 | 0-1 |
| Water | ad 100 | ad 100 | ad 100 | ad 100 | ad 100 | ad 100 | ad 100 | ad 100 | ad 100 | ad 100 |

1.-7. (canceled)

8. A cosmetic or dermatological preparation, wherein the preparation comprises at least one of benzethonium chloride, methylisothiazolinone, piroctone olamine, and lauroyl ethyl arginate, and does not contain a preservative that comprises a phenolic group.

9. The preparation of claim 8, wherein the preparation does not contain parabens.

10. The preparation of claim 8, wherein the preparation does not contain phenoxyethanol.

11. The preparation of claim 8, wherein the preparation comprises at least two of benzethonium chloride, methylisothiazolinone, piroctone olamine, and lauroyl ethyl arginate.

12. The preparation of claim 8, wherein the preparation comprises at least three of benzethonium chloride, methylisothiazolinone, piroctone olamine, and lauroyl ethyl arginate.

13. The preparation of claim 8, wherein the preparation comprises benzethonium chloride, methylisothiazolinone, piroctone olamine, and lauroyl ethyl arginate.

14. The preparation of claim 8, wherein the preparation comprises a total of from 0.01% to 5% by weight of the at least one of benzethonium chloride, methylisothiazolinone, piroctone olamine, and lauroyl ethyl arginate, based on a total weight of the preparation.

15. The preparation of claim 14, wherein the preparation comprises a total of from 0.1% to 2% by weight of the at least one of benzethonium chloride, methylisothiazolinone, piroctone olamine, and lauroyl ethyl arginate.

16. The preparation of claim 11, wherein the preparation comprises a total of from 0.01% to 5% by weight of the at least two of benzethonium chloride, methylisothiazolinone, piroctone olamine, and lauroyl ethyl arginate, based on a total weight of the preparation.

17. The preparation of claim 16, wherein the preparation comprises a total of from 0.1% to 2% by weight of the at least two of benzethonium chloride, methylisothiazolinone, piroctone olamine, and lauroyl ethyl arginate.

18. The preparation of claim 12, wherein the preparation comprises a total of from 0.01% to 5% by weight of the at least three of benzethonium chloride, methylisothiazolinone, piroctone olamine, and lauroyl ethyl arginate, based on a total weight of the preparation.

19. The preparation of claim 18, wherein the preparation comprises a total of from 0.1% to 2% by weight of the at least three of benzethonium chloride, methylisothiazolinone, piroctone olamine, and lauroyl ethyl arginate.

20. The preparation of claim 13, wherein the preparation comprises a total of from 0.01% to 5% by weight of benzethonium chloride, methylisothiazolinone, piroctone olamine, and lauroyl ethyl arginate, based on a total weight of the preparation.

21. The preparation of claim 20, wherein the preparation comprises a total of from 0.1% to 2% by weight of benzethonium chloride, methylisothiazolinone, piroctone olamine, and lauroyl ethyl arginate.

22. The preparation of claim 8, wherein the preparation is suitable for at least one of cleansing skin, cleansing hair, and caring for hair and comprises at least one of benzethonium chloride and lauroyl ethyl arginate.

23. The preparation of claim 22, wherein the preparation comprises benzethonium chloride and lauroyl ethyl arginate.

24. The preparation of claim 8, wherein the preparation is free of any other preservatives.

25. The preparation of claim 13, wherein the preparation is free of any other preservatives.

26. The preparation of claim 21, wherein the preparation is free of any other preservatives.

27. A method of improving the biofilm-inhibiting effectiveness of a cosmetic or dermatological preparation which is free of preservatives having a phenolic group, wherein the method comprises including in the preparation at least one of benzethonium chloride, methylisothiazolinone, piroctone olamine, and lauroyl ethyl arginate.
